(19) United States
(12) Patent Application Publication
(10) Pub. No.: US 2023/0005605 A1
(43) Pub. Date: Jan. 5, 2023

(54) INTERNAL BENCHMARKING OF CURRENT OPERATIONAL WORKFLOW PERFORMANCES OF A HOSPITAL DEPARTMENT

(71) Applicant: KONINKLIJKE PHILIPS N.V., EINDHOVEN (NL)

(72) Inventors: RANJITH NAVEEN TELLIS, TEWKSBURY, MA (US); OLGA STAROBINETS, NEWTON, MA (US); USHANANDINI RAGHAVAN, LEXINGTON, NY (US); PRESCOTT PETER KLASSEN, CAMBRIDGE, MA (US)

(21) Appl. No.: 17/781,072

(22) PCT Filed: Dec. 4, 2020
(86) PCT No.: PCT/EP2020/084565

§ 371 Date: May 31, 2022

Related U.S. Application Data
(60) Provisional application No. 62/947,559, filed on Dec. 13, 2019.

(51) Int. Cl. G16H 40/20; G06Q 10/06

(52) U.S. Cl. CPC G16H 40/20; G06Q 10/06393; G06Q 10/0633; G06Q 10/067

(57) ABSTRACT

An apparatus (10) for generating benchmarking metrics of current operational workflow performance of a hospital department includes at least one electronic processor (20) programmed to: generate a department profile (34) identifying resources of the hospital department including at least an active medical equipment inventory and a personnel profile; retrieve current statistics for the hospital department including at least one of patient arrival timeliness, patient no-show, and emergency department (ED) arrival statistics; compute values of one or more key performance indicator (KPI) metrics (40) for the current statistics; generate an executable workflow model (44) for workflow processes of the hospital department including temporal aspects of the workflow processes, the workflow model having variables representing at least patient arrival timeliness, patient no-show, and ED arrival; simulate a best case scenario (50) by executing the workflow model on inputs including the department profile and best case values for the variables of the workflow model and compute values of the one or more KPI metrics for the simulated best case scenario; simulate a worst case scenario (52) by executing the workflow model on inputs including the department profile and worst case values for the variables of the workflow model and compute values of the one or more KPI metrics for the simulated worst case scenario; and output, on at least one display device (24), the values of the one or more KPI metrics computed for the simulated best case scenario, the values of the one or more KPI metrics computed for the simulated worst case scenario, and the values of the one or more KPI metrics computed for the current statistics.

### FIELD

The following relates generally to the hospital department management arts, hospital department benchmarking arts, hospital workflow assessment and improvement arts, hospital workflow simulation arts, and related arts.

### BACKGROUND

Healthcare institutions seeking to assess performance of a hospital department typically seek to benchmark operational efficiency of the department against similar hospital departments at other institutions. Such benchmarking can be used to determine how well their competitors are doing, and to assess whether there is scope for improvement in operational efficiency.

However, a meaningful comparison would require finding at least one, and preferably two, three, or even more, similar institutions (e.g., having a similar patient case mix, number of resources, etc.) to compare against. It is, however, difficult, to find such a similar institution. Even if potentially similar hospital departments are identified at other institutions, it is challenging to gain access to operational performance data of other institutions. This can be due to unwillingness to share information for competitive reasons, and/or inability to share information that may contain personally identifying information (PII) or protected health information (PHI) about patients. For example, in the United States, the Health Insurance Portability and Accountability Act (HIPAA) constrains disclosure of patient PII.

The following discloses certain improvements to overcome these problems and others.

### SUMMARY

In one aspect, an apparatus for generating benchmarking metrics of current operational workflow performance of a hospital department includes at least one electronic processor programmed to: generate a department profile identifying resources of the hospital department including at least an active medical equipment inventory and a personnel profile; retrieve current statistics for the hospital department including at least one of patient arrival timeliness, patient no-show, and emergency department (ED) arrival statistics; compute values of one or more key performance indicator (KPI) metrics for the current statistics; generate an executable workflow model for workflow processes of the hospital department including temporal aspects of the workflow processes, the workflow model having variables representing at least patient arrival timeliness, patient no-show, and ED arrival; simulate a best case scenario by executing the workflow model on inputs including the department profile and best case values for the variables of the workflow model and compute values of the one or more KPI metrics for the simulated best case scenario; simulate a worst case scenario by executing the workflow model on inputs including the department profile and worst case values for the variables of the workflow model and compute values of the one or more KPI metrics for the simulated worst case scenario; and output, on at least one display device, the values of the one or more KPI metrics computed for the simulated best case scenario, the values of the one or more KPI metrics computed for the simulated worst case scenario, and the values of the one or more KPI metrics computed for the current statistics.

In another aspect, a non-transitory computer readable medium stores instructions executable by at least one electronic processor to perform a method for generating benchmarking metrics of current operational workflow performance of a hospital department. The method includes: generating a department profile identifying resources of the hospital department including at least an active medical equipment inventory and a personnel profile by operations including: retrieving hospital department data and patient data from at least one database; generating a department profile for each resource from the retrieved hospital department data and patient data; and providing a user interface via which the generated profiles are displayed on the at least one display device and via which a user can modify the generated profiles using via at least one user input device; retrieving current statistics for the hospital department including at least one of patient arrival timeliness, patient no-show, and ED arrival statistics; computing values of one or more KPI metrics for the current statistics; generating an executable workflow model for workflow processes of the hospital department including temporal aspects of the workflow processes, the workflow model having variables representing at least patient arrival timeliness, patient no-show, and ED arrival; simulating a best case scenario by executing the workflow model on inputs including the department profile and best case values for the variables of the workflow model and compute values of the one or more KPI metrics for the simulated best case scenario; simulating a worst case scenario by executing the workflow model on inputs including the department profile and worst case values for the variables of the workflow model and compute values of the one or more KPI metrics for the simulated worst case scenario; and outputting, on at least one display device, the values of the one or more KPI metrics computed for the simulated best case scenario, the values of the one or more KPI metrics computed for the simulated worst case scenario, and the values of the one or more KPI metrics computed for the current statistics.

In another aspect, a method for generating benchmarking metrics of current operational workflow performance of a hospital department includes: generating a department profile identifying resources of the hospital department including at least an active medical equipment inventory and a personnel profile; retrieving current statistics for the hospital department including at least patient arrival timeliness, patient no-show, and ED arrival statistics; computing values of one or more KPI metrics for the current statistics; generating an executable workflow model for workflow processes of the hospital department including temporal aspects of the workflow processes, the workflow model having random variables representing at least one of patient arrival timeliness, patient no-show, and ED arrival; simulating a best case scenario by executing the workflow model using Monte Carlo simulation on inputs including the department profile and best case values for the random variables of the workflow model and compute values of the one or more KPI metrics for the simulated best case scenario; simulating a worst case scenario by executing the workflow model using Monte Carlo simulation on inputs including the department profile and worst case values for the random variables of the workflow model and compute values of the one or more KPI metrics for the simulated worst case scenario; and outputting, on at least one display device, the values of the one or more KPI metrics computed for the simulated best case scenario, the values of the one or more KPI metrics computed for the simulated worst case scenario, and the values of the one or more KPI metrics computed for the current statistics.

One advantage resides in providing a benchmarking procedure for a hospital department using data only from the hospital department.

Another advantage resides in providing a benchmarking procedure for a hospital department without relying on data from external hospital departments.

Another advantage resides in providing a hospital department with a representation of best- and worst-case scenarios for workflows in the hospital department, and optionally also one or more intermediate-case scenarios.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for generating benchmarking metrics of current operational workflow performance of a hospital department in accordance with the present disclosure.

FIG. 2 shows example flowchart operations performed by the apparatus of FIG. 1.

FIG. 3 shows an example of benchmarking outputs displayed on the apparatus of FIG. 1.

### DETAILED DESCRIPTION

To perform quality control of a hospital department, it is common practice to compare with performance of similarly situated departments at other medical institutions. However, the data for other medical institutions needed for the benchmarking may be difficult to obtain due to competitive and/or patient privacy considerations.

The following discloses a benchmarking approach referred to as “internal” benchmarking, in which the performance data of the hospital department being evaluated is extrapolated to best case and worst case scenarios (and one or more intermediate scenarios) and the current performance is compared with these extrapolations. This permits benchmarking without the need for data from other medical institutions.

In some embodiments disclosed herein, an Electronic Medical Record (EMR) or other hospital database is mined to determine relevant information, including: the number of imaging systems (of each imaging modality, in the case of a multimodality department) and statistics such as the types of imaging examinations being performed, rate of late arrivals, rate of no-shows, rate of unscheduled ED arrivals, and so forth. The number of imaging technicians may be mined from department human resources (HR) databases or other sources. This provides a department profile, which may be presented to a hospital administrator for review and (if needed) manual adjustment.

A patient workflow model is also generated for the department (and for each imaging modality, if multimodality). The patient workflow model can be a Business Process Model (BPM) or other dynamic model that captures the process flow and temporal aspects. In the illustrative example, each step of the process is characterized by activity, location, resources utilized, and time interval for the activity (with the time interval, and possibly the resources used, being expressed stochastically). To generate the model, a standard template model may be employed, which may be adjusted to the specifics of the hospital department using statistical data mined from the EMR and/or information obtained from interviews with department staff or other information sources.

A given scenario is analyzed by Monte Carlo simulations. Each simulation receives as input a schedule of patients scheduled for respective imaging examinations with various arrival times (on-time, some late, etc.) and some ED arrivals and/or no-shows, generated stochastically in accord with the mined department profile. The stochastic generation process can be biased to reflect a “best case” scenario by biasing the stochastic generation to produce most patients arriving on-time and on-schedule with few or no ED arrivals or no-shows. The stochastic generation process can be biased to reflect a “worst case” scenario by biasing the stochastic generation to produce many late arrivals and numerous ED arrivals and no-shows. Various intermediate scenarios can also be generated. Each stochastically generated schedule is “processed” by running the patients on the schedule through the patient workflow model, and computing key performance indicator (KPI) metrics.

The KPIs for the simulated scenarios are then compared with actual current statistics for the hospital department. For example, the best case scenario provides an upper limit on the number of patients that could realistically be handled per day, the worst case scenario provides a lower limit on the number of patients that could realistically be handled per day, and these can be compared against the number of patients actually handled per day. Similar comparisons can be run for patient wait time, scanner utilization, and other KPIs.

While described in the context of medical imaging departments, the disclosed concept can be applied more generally to any hospital department that processes patients in accordance with workflows that are amenable to modeling.

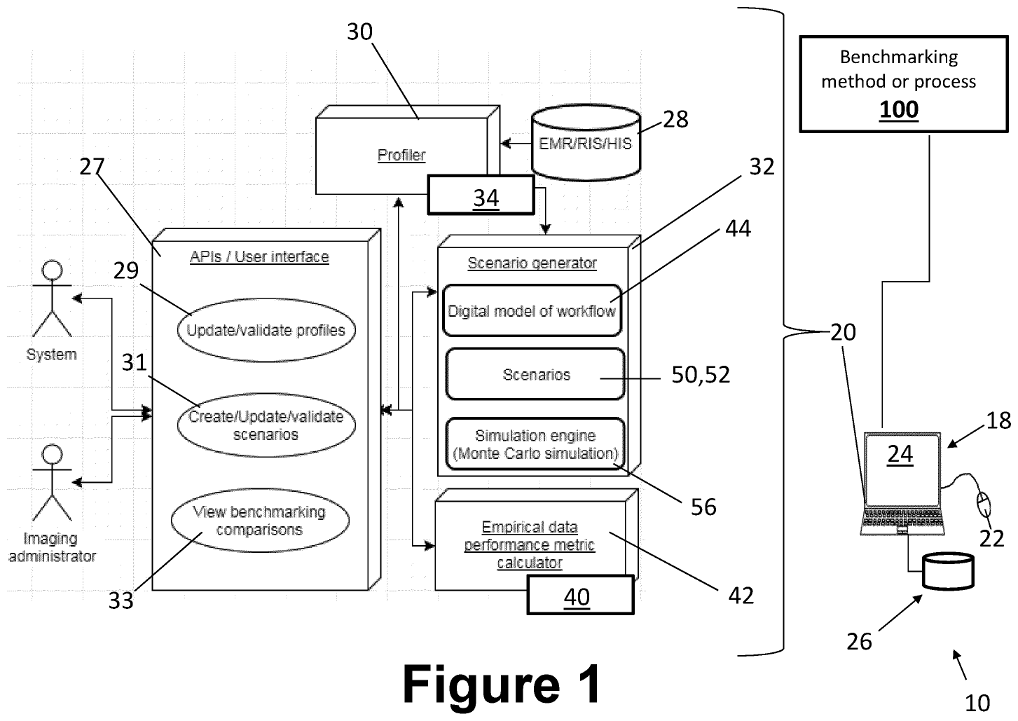

With reference to FIG. 1, an illustrative apparatus 10 for generating benchmarking metrics of current operational workflow performance of a hospital department is shown. FIG. 1 also shows an electronic processing device 18, such as a workstation computer, or more generally a computer. Alternatively, the electronic processing device 18 can be embodied as a server computer or a plurality of server computers, e.g. interconnected to form a server cluster, cloud computing resource, or so forth. The workstation 18 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 18.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of an graphical user interface (GUI) or application program interface (API) 27 for display on the display device 24.

The apparatus 10 also includes, or is otherwise in operable communication with, one or more databases 28 storing, for example, data related to patients of the hospital department, data related to workflows of the hospital department, and so forth. The database 28 can be any suitable database, including a Radiology Information System (RIS) database, a Picture Archiving and Communication System (PACS) database, an Electronic Medical Records (EMR) database, a Health Information System (HIS) database, and so forth. Alternatively, the database 28 can be implemented in the non-transitory medium or media 26. The workstation 18 can be used to access the stored data

FIG. 1 also shows modules programmed into the at least one electronic processor 20. For example, FIG. 1 shows a profiler module 30 and a scenario generator module 32. The profiler module 30 is programmed to create profiles 34 for several aspects of the hospital department, including resources (e.g., medical devices, medical staff, and so forth), patients, work orders, department policies, and so forth. The profiles 34 are used to estimate the amount of work the resources is capable of producing under different settings and/or scenarios. The data related to these aspects can be stored in the database 28 and/or the non-transitory computer readable medium 26, or can be manually entered by a user via the at least one user input device 22 (e.g., such as answers to a patient screening form, results from a radiology report, and so forth). Furthermore, to calculate KPI metrics for the actual statistical data for the hospital department, an empirical data performance metric calculator module 42 is programmed into the at least one electronic processor 20.

The apparatus 10 is configured as described above to perform method or process 100 for generating benchmarking metrics of current operational workflow performance of a hospital department. The non-transitory storage medium 26 stores instructions which are readable and executable by the at least one electronic processor 20 to perform disclosed operations including performing the method or process 100 for generating benchmarking metrics of current operational workflow performance of a hospital department. In some examples, the method 100 may be performed at least in part by cloud processing.

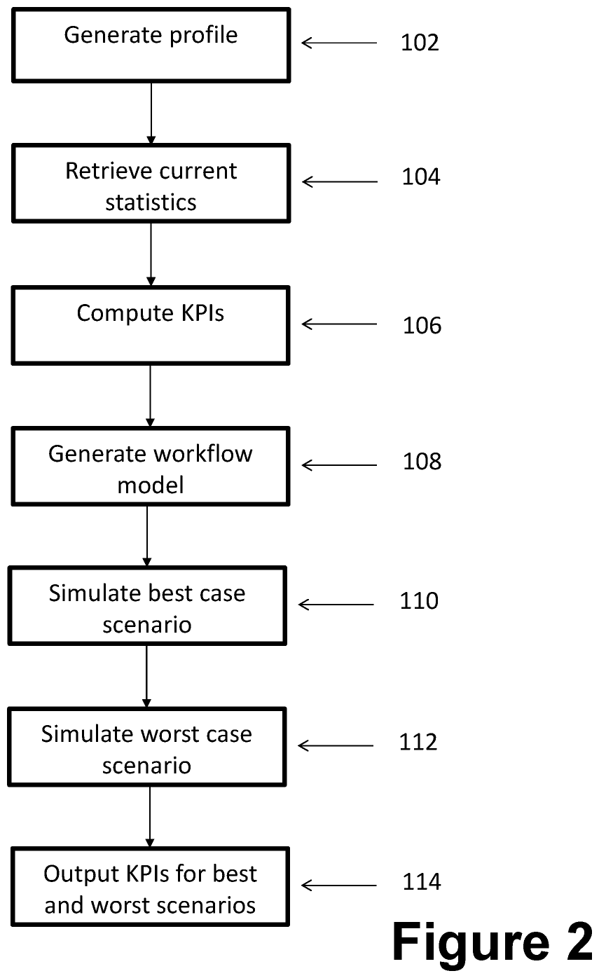

With continuing reference to FIG. 1, and with reference to FIG. 2, an illustrative embodiment of the method 100 generating benchmarking metrics of current operational workflow performance of a hospital department is diagrammatically shown as a flowchart. At an operation 102, a department profile 34 that identifies resources of the hospital department is generated. The resources can include at least, for example, an active medical equipment inventory, which refers to equipment that is in use, and not equipment that is not in use (excluding temporary outages). In one example, for an imaging hospital department, the active medical equipment inventory can be an inventory of active medical imaging devices annotated at least by imaging modality. In another example, for an hematology hospital department, the active medical equipment inventory can include an inventory of hematology analyzer labeled as to, for example, type or types of analyses each analyzer can perform. In a further example, for a surgical laboratory hospital department, the active medical equipment inventory can include surgical devices or suites with information such as availability of oxygen or anesthetic lines, specified medical equipment, or so forth. These are merely non-limiting examples. The department profile 34 can also include, for example, a personnel profile that can include, for example, a list of employees assigned to the department with each employee identified as to role and impacting the workflow, such as imaging technicians and possibly patient transport personnel.

To generate the department profile 34, data can be retrieved from the at least one database 28 and/or the non-transitory computer readable medium 26. For example, hospital data can be retrieved, and include one or more of types of procedures to be performed, distribution of process time for each procedure, availability of resources, a maintenance schedule for the resources, downtime of the resources, a resource to staff ratio, a patient to nurse ratio, an order list of procedures, scheduled examinations, and unscheduled examinations. Patient data can also be retrieved, including one or more of a type of patient, records of no-shows or late arrivals to appointments, age, gender, need for sedation, presence of contrast allergies, presence of claustrophobia, and presence of foreign bodies. From the retrieved data, a department profile 34 can be generated for each resource (e.g., each piece of active medical equipment and each personnel).

The profiles 34 for each aspect can be displayed on the display device 24 via the GUI 27 for review by a user and/or medication of the profiles via the at least one user input device 22. To do so, the user can select an “update/validate profiles” field 29 implemented in the GUI 27. The profiles 34 can display relevant information to be extracted for the scenario generator module 32. For example, a profile 34 for an imaging scanner can contain the types of procedures, the distribution of process time for each of the scan types, its availability, a maintenance schedule, a model to predict downtime, etc. In another example, a profile 34 for the hospital department staff can contain staff availability, skill level, vacation plans, a model to predict absences, etc. In a further example, a profile 34 for patients can include types of patients (e.g., inpatient, outpatient, ED, and so forth), a model to predict probability of no-show/late arrivals, ages, genders, need for sedation, contrast allergies, claustrophobic presences, foreign bodies presences, etc. In yet another example, a department profile 34 can include a device to staff ratio, a patient to nurse ratio, and other department policies. In another example, a work order profile 34 can include an order mix, scheduled/unscheduled exams to determine short-term demand, etc. These are merely non-limiting examples, and are not to be construed as limiting. However, more relevant information extracted from the profiles 34 can include: procedure rooms and devices in those rooms (e.g., imaging modalities in respective procedure rooms); times for procedures types; types of patients; types of work orders per room and staff member(s); procedure room availability and schedules; staff availability and schedules; a number of procedures that the rooms can handle; and preferred time slots for certain types of exams.

At an operation 104, current statistics for the hospital department are retrieved (e.g., from the at least one database 28 and/or the non-transitory computer readable medium 26). The current statistics for the hospital department can include, for example, at least patient arrival timeliness, patient no-show, and ED arrival statistics.

At an operation 106, values of one or more KPI metrics 40 can be computed for the current statistics. To do so, the empirical data performance metric calculator module 42 performs the calculations.

At an operation 108, an executable workflow model 44 is generated for workflow processes of the hospital department. The workflow model 44 can include information such as, for example, temporal aspects of the workflow processes in the hospital department. The workflow model 44 can include variables, such as scalar variables, or a random variable with a probability density function (PDF) value, such as statistical distributions. The variables can include at least one of patient arrival timeliness, patient no-show, and ED arrival, for example. To generate the workflow model 44, a mode workflow template is retrieved (e.g., from the non-transitory computer readable medium 26) and adjusted based on the computed current statistics. In some examples, the workflow model 44 can be a Business Process Model (BPM), or any other suitable model such as a queuing model, a discrete event simulation (DES) model, and so forth.

At an operation 110, a “best-case” scenario 50 is simulated by executing the workflow model 44. Inputs, such as the department profile 34 and “best case values” for the variables of workflow model 44, are input to the workflow model. The best case values can include, for example, values representing: patient arrival timeliness that is better than the current statistics; patient no-shows that are lower than the current statistics; ED arrivals that are lower than the current statistics; patients being on-time; no patients being no-shows; no ED values, and so forth. The scenario generator module 32 then executes the workflow model 44 to simulate the best case scenario 50 with these variables. Values of one or more KPI metrics 40 can be calculated for the simulated best case scenario 50 using the calculator module 42.

Similarly, at an operation 112, a “worst case” scenario 52 is simulated by executing the workflow model 44. The operation 112 can be performed concurrently with, or subsequently to, the operation 110. The department profile 34 and “worst case values” for the variables of the workflow model 44 are input to the model. The worst case values can include, for example, values representing: patient arrival timeliness that is worse than the current statistics; patient no-shows that are higher than the current statistics; ED arrivals that are higher than the current statistics; no patients being on time, and so forth. The scenario generator module 32 then executes the workflow model 44 to simulate the worst case scenario 52 with these variables. Values of one or more KPI metrics 40 can be calculated for the simulated worst case scenario 52 using the calculator module 42. In some examples, at least one “intermediate” scenario 54 can be simulated, e.g., using the department profile 34 and intermediate values between the best and worst case variable values as described. One or more KPI metrics 40 can be calculated for the intermediate scenario(s) 54.

In one example embodiment, the variables of the workflow model 44 are random variables, which are used to instantiate a Monte Carlo simulator 56 implemented in the scenario generator module 32. The workflow model 44 is executed using the Monte Carlo simulator 56. To do so, for each simulated patient the Monte Carlo simulator 56 draws a scalar value for each random variable from its corresponding PDF. For example, if a random variable is selected representing patient no-shows, this random variable may have a PDF that is 90% “shows” and 10% “no shows”. For a given patient, the draw of the scalar value would be a show for 90% of each Monte Carlo simulated patient, and a no show for 10% of the Monte Carlo simulated patients. In another example, for a random variable of patient timeliness, the PDF can be a Gaussian distribution that is peaked at on-time values and tails off with increasing lateness. For each patient, the lateness value is drawn from the corresponding PDF.

At an operation 112, the values of the KPI metrics 40 for the simulated best case scenario 50 and the simulated worst case scenario 52 (and, where computed, for the intermediate scenario(s) 56), along with values of KPI metrics computed for the current statistics are output on the at least one display device 24 via the GUI 27. In addition to the update/validate profiles field 29, the GUI 27 also includes a “create/update/validate/scenarios” field 31 which the user can use to execute the scenario generator module 32. In addition, a “view benchmarking comparison” field 33 is included, which the user can access to compare the values of the KPI metrics 40 with the current statistics for the hospital department.

In a particular example, the hospital department can be a medical imaging department. Once the corresponding profile(s) 34 are generated, the scenario generator module 32 simulates the best case scenario 50 and the worst case scenario 52. For example, the medical imaging department can include 2 magnetic resonance (MR) scanners, one of which is a 1.5 Tesla (T) model and the other is a 3T model, and the department as a sufficient number of staff. The best case scenario 50 can include, for example, handling 22-24 imaging exams, with a patient wait time PDF of p(w), staff utilization PDF of p(s), scanner utilization PDF of p(m), over time PDF of p(ot), and so forth.

In another particular example, the scenario generator module 32 is programmed to update the best case scenario 50 and/or the worst case scenario 52 based on one or more changing scenarios. For example, the user can select the “view benchmarking comparison” field 33 on the GUI 27 to view the values of the KPI metrics 40. The values of the KPI metrics 40 are compared against the best case scenario 50 and/or the worst case scenario 52. The user can then input, via the at least one user input device 22, one or more changes to the workflow model 44 to update or change the best case scenario 50 and/or the worst case scenario 52 (which would also update the values of the KPI metrics 40 in a downstream approach). The user can then compare the updated values of the KPI metrics 40 based on the input changes to the best case scenario 50 and/or the worst case scenario 52 to determine if a predetermined change threshold is satisfied (e.g., if a desired change has occurred). The desired change can be observed based on the updated values of the KPI metrics 40. If the updated values are closer to a desired range or value for the user, then the desired change has been achieved.

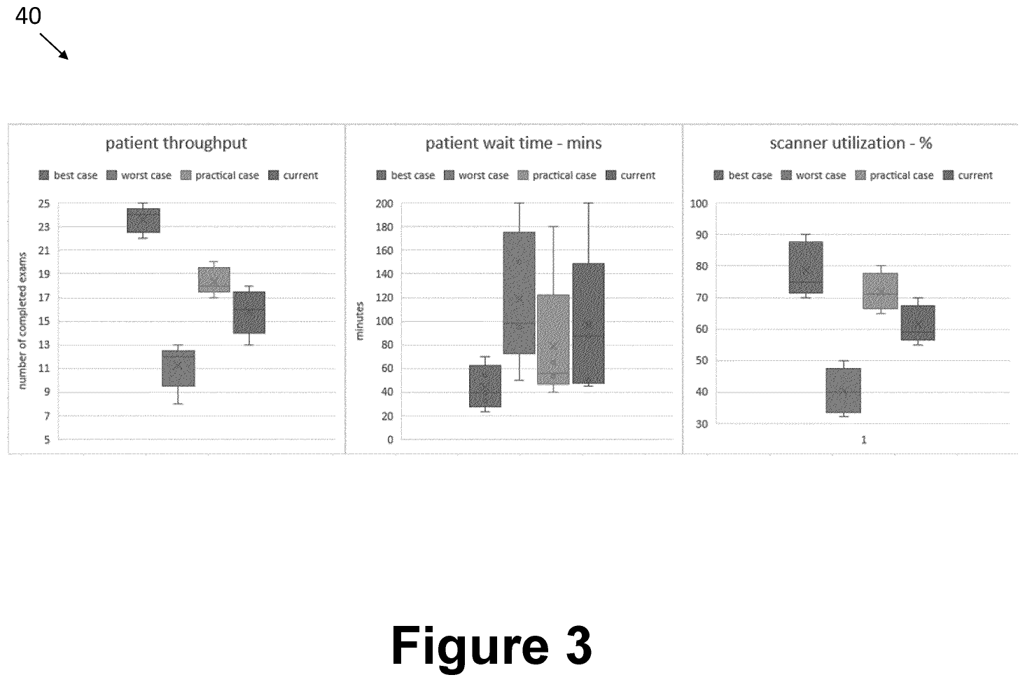

FIG. 3 shows an example of the output values of the KPI metrics 40 as a graphical representation on the display device 24 via the GUI 27. The KPI metrics 40 shown in FIG. 3 include a “patient throughput” KPI metric, a patient wait time (in minutes) KPI metric, and a scanner utilization (in percentage) KPI metric. As shown in FIG. 3, the current throughput (left) of a scanner is on average 16 exams/day with average patient wait time of 95 mins (center) and average scanner utilization (right) of 62%. The current performance of the system is better than a worse case, but can be closer to the practical case scenario where the daily throughput should have been, for example, on average 18 exams/day with average patient wait time close to 80 mins and average scanner utilization around 72%.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus for generating benchmarking metrics of current operational workflow performance of a hospital department, the apparatus including at least one electronic processor programmed to:
generate a department profile identifying resources of the hospital department including at least an active medical equipment inventory and a personnel profile;
retrieve current statistics for the hospital department including at least one of patient arrival timeliness, patient no-show, and emergency department (ED) arrival statistics;
compute values of one or more key performance indicator (KPI) metrics for the current statistics;
generate an executable workflow model for workflow processes of the hospital department including temporal aspects of the workflow processes, the workflow model having variables representing at least patient arrival timeliness, patient no-show, and ED arrival;
simulate a best case scenario by executing the workflow model on inputs including the department profile and best case values for the variables of the workflow model and compute values of the one or more KPI metrics for the simulated best case scenario;
simulate a worst case scenario by executing the workflow model on inputs including the department profile and worst case values for the variables of the workflow model and compute values of the one or more KPI metrics for the simulated worst case scenario; and
output, on at least one display device, the values of the one or more KPI metrics computed for the simulated best case scenario, the values of the one or more KPI metrics computed for the simulated worst case scenario, and the values of the one or more KPI metrics computed for the current statistics.

2. The apparatus of claim 1, wherein:
the best case values for the variables of the workflow model include values representing patient arrival timeliness that is better than the current statistics, patient no-shows that are lower than the current statistics, and ED arrivals that are lower than the current statistics, and
the worst case values for the variables of the workflow model include values representing patient arrival timeliness that is worse than the current statistics, patient no-shows that are higher than the current statistics, and ED arrivals that are higher than the current statistics.

3. The apparatus of claim 1, wherein:
the best case values for the variables of the workflow model include values representing all patients being on-time; and
the worst case values for the variables of the workflow model include values representing no patients being on-time.

4. The apparatus of claim 1, wherein:
the best case values for the variables of the workflow model include values representing no patients being no-shows.

5. The apparatus of claim 1, wherein:
the best case values for the variables of the workflow model include values representing no ED arrivals.

6. The apparatus of claim 1, wherein the at least one electronic processor is further programmed to:
simulate at least one intermediate scenario by executing the workflow model on inputs including the department profile and intermediate values for the variables of the workflow model that are intermediate between the best case scenario and the worst case scenario, and compute values of the one or more KPI metrics for the simulated intermediate scenario; and
further outputting, on at least one display device, the values of the one or more KPI metrics computed for the simulated at least one intermediate scenario.

7. The apparatus of claim 1, wherein the at least one electronic processor is programmed to generate the department profile by operations including:
retrieving hospital department data and patient data from at least one database, the hospital department data including one or more of types of procedures to be performed, distribution of process time for each procedure, availability of resources, a maintenance schedule for the resources, downtime of the resources, a resource to staff ratio, a patient to nurse ratio, an order list of procedures, scheduled examinations, and unscheduled examinations, the patient data including one or more of a type of patient, records of no-shows or late arrivals to appointments, age, gender, need for sedation, presence of contrast allergies, presence of claustrophobia, and presence of foreign bodies;
generating a department profile for each resource from the retrieved hospital department data and patient data; and
providing a user interface via which the generated profiles are displayed on the at least one display device and via which a user can modify the generated profiles using via at least one user input device.

8. The apparatus of claim 1, wherein the at least one electronic processor is programmed to generate the workflow model by operations including:
retrieving a model workflow template;
adjusting the model workflow template based on the current statistics for the hospital department.

9. The apparatus of claim 1, wherein the at least one electronic processor is programmed to:
receive one or more user inputs indicative of a change one or more values of the workflow model to update at least one of the best case scenario and the worst case scenario;
compare one or more updated values of the KPI metrics resulting from updating the update at least one of the best case scenario and the worst case scenario with previously-obtained value of KPI metrics; and
update the workflow model when the updated values of the KPI metrics satisfy a predetermined update threshold.

10. The apparatus of claim 1, wherein the variables of the workflow model include random variables, and the at least one electronic processor is programmed to execute the workflow model on inputs including the random variables instantiated using Monte Carlo simulation.

11. The apparatus of claim 1, wherein the hospital department is a medical imaging department and the active medical equipment inventory comprises an inventory of active medical imaging devices annotated at least by imaging modality.

12. A non-transitory computer readable medium storing instructions executable by at least one electronic processor to perform a method for generating benchmarking metrics of current operational workflow performance of a hospital department, the method including:
generating a department profile identifying resources of the hospital department including at least an active medical equipment inventory and a personnel profile by operations including:
retrieving hospital department data and patient data from at least one database;
generating a department profile for each resource from the retrieved hospital department data and patient data; and
providing a user interface via which the generated profiles are displayed on the at least one display device and via which a user can modify the generated profiles using via at least one user input device;
retrieving current statistics for the hospital department including at least one of patient arrival timeliness, patient no-show, and emergency department (ED) arrival statistics;
computing values of one or more key performance indicator (KPI) metrics for the current statistics;
generating an executable workflow model for workflow processes of the hospital department including temporal aspects of the workflow processes, the workflow model having variables representing at least patient arrival timeliness, patient no-show, and ED arrival;
simulating a best case scenario by executing the workflow model on inputs including the department profile and best case values for the variables of the workflow model and compute values of the one or more KPI metrics for the simulated best case scenario;
simulating a worst case scenario by executing the workflow model on inputs including the department profile and worst case values for the variables of the workflow model and compute values of the one or more KPI metrics for the simulated worst case scenario; and
outputting, on at least one display device, the values of the one or more KPI metrics computed for the simulated best case scenario, the values of the one or more KPI metrics computed for the simulated worst case scenario, and the values of the one or more KPI metrics computed for the current statistics.

13. The non-transitory computer readable medium of claim 12, wherein:
the best case values for the variables of the workflow model include values representing patient arrival timeliness that is better than the current statistics, patient no-shows that are lower than the current statistics, and ED arrivals that are lower than the current statistics, and
the worst case values for the variables of the workflow model include values representing patient arrival timeliness that is worse than the current statistics, patient no-shows that are higher than the current statistics, and ED arrivals that are higher than the current statistics.

14. The non-transitory computer readable medium of claim 12, wherein generating the workflow model includes:
retrieving a model workflow template;
adjusting the model workflow template based on the current statistics for the hospital department.

15. The non-transitory computer readable medium of claim 12, wherein the variables of the workflow model include random variables, and the method further includes:
executing the workflow model on inputs including the random variables instantiated using Monte Carlo simulation.

16. The non-transitory computer readable medium of claim 12, wherein:
the hospital department data includes one or more of types of procedures to be performed, distribution of process time for each procedure, availability of resources, a maintenance schedule for the resources, downtime of the resources, a resource to staff ratio, a patient to nurse ratio, an order list of procedures, scheduled examinations, and unscheduled examinations; and
the patient data includes one or more of a type of patient, records of no-shows or late arrivals to appointments, age, gender, need for sedation, presence of contrast allergies, presence of claustrophobia, and presence of foreign bodies.

17. A method for generating benchmarking metrics of current operational workflow performance of a hospital department, the method including:
generating a department profile identifying resources of the hospital department including at least an active medical equipment inventory and a personnel profile;
retrieving current statistics for the hospital department including at least one of patient arrival timeliness, patient no-show, and emergency department (ED) arrival statistics;
computing values of one or more key performance indicator (KPI) metrics for the current statistics;
generating an executable workflow model for workflow processes of the hospital department including temporal aspects of the workflow processes, the workflow model having random variables representing at least patient arrival timeliness, patient no-show, and ED arrival;
simulating a best case scenario by executing the workflow model using Monte Carlo simulation on inputs including the department profile and best case values for the random variables of the workflow model and compute values of the one or more KPI metrics for the simulated best case scenario;
simulating a worst case scenario by executing the workflow model using Monte Carlo simulation on inputs including the department profile and worst case values for the random variables of the workflow model and compute values of the one or more KPI metrics for the simulated worst case scenario; and
outputting, on at least one display device, the values of the one or more KPI metrics computed for the simulated best case scenario, the values of the one or more KPI metrics computed for the simulated worst case scenario, and the values of the one or more KPI metrics computed for the current statistics.

18. The method of claim 17, wherein:
the best case values for the variables of the workflow model include values representing at least: patient arrival timeliness that is better than the current statistics, patient no-shows that are lower than the current statistics, ED arrivals that are lower than the current statistics, all patients being on-time, no patients being no-shows, and no ED arrivals; and
the worst case values for the variables of the workflow model include values representing at least: patient arrival timeliness that is worse than the current statistics, patient no-shows that are higher than the current statistics, ED arrivals that are higher than the current statistics, and no patients being on-time.

19. The method of claim 17, wherein generating the department profile includes:
retrieving hospital department data and patient data from at least one database, the hospital department data including one or more of types of procedures to be performed, distribution of process time for each procedure, availability of resources, a maintenance schedule for the resources, downtime of the resources, a resource to staff ratio, a patient to nurse ratio, an order list of procedures, scheduled examinations, and unscheduled examinations, the patient data including one or more of a type of patient, records of no-shows or late arrivals to appointments, age, gender, need for sedation, presence of contrast allergies, presence of claustrophobia, and presence of foreign bodies;
generating a department profile for each resource from the retrieved hospital department data and patient data; and
providing a user interface via which the generated profiles are displayed on the at least one display device and via which a user can modify the generated profiles using via at least one user input device.

20. The method of claim 17, wherein generating the workflow model includes:
retrieving a model workflow template;
adjusting the model workflow template based on the current statistics for the hospital department.
